(19) United States
(12) Patent Application Publication
(10) Pub. No.: US 2021/0070507 A1
(43) Pub. Date: Mar. 11, 2021

(54) Container Cap

(71) Applicant: Keymed (Medical & Industrial Equipment) Ltd., Essex (GB)

(72) Inventors: Adam Arnold, Essex (GB); Jordan Jowitt, Essex (GB)

(73) Assignee: Keymed (Medical & Industrial Equipment) Ltd., Esex (GB)

(21) Appl. No.: 17/018,526

(22) Filed: Sep. 11, 2020

(30) Foreign Application Priority Data
Sep. 11, 2019 (GB) 1913095.4

(51) Int. Cl. B65D 47/06; A61M 1/00

(52) U.S. Cl. CPC B65D 47/06; B65D 2547/063; A61M 1/0001

(57) ABSTRACT

A cap for a container holding a liquid for medical use comprises a lid portion and an aperture for receiving a tube. The aperture comprises a slot with first and second ends. The width of the slot is smaller than the length between the first and second ends and the slot tapers from the first end towards the second end. Preferably, a gripping feature such as a protruding rim around the perimeter of the aperture is provided to engage the surface of a tube passing through the aperture.

### CROSS-REFERENCE TO RELATED APPLICATIONS

This patent application claims the benefit of United Kingdom Patent Application No. GB1913095.4, filed Sep. 11, 2019, which is incorporated herein by reference in its entirety for all purposes.

### BACKGROUND OF THE INVENTION

The present invention relates to a cap for bottle or other a container holding fluid which is intended to be used in a medical procedure, for example irrigation fluid for use in an endoscopy.

For such applications, a typical container is fitted with a lid having a hole through which a flexible tube is passed. Liquid is withdrawn from the bottle through the tube by a pump, such as a peristaltic pump. Tubes of different diameter may be used for different applications. The tube needs to be fitted easily through the lid, and easily removed, but should be secure when in place. Otherwise, vibrations from the pump may cause the tube to move, potentially rising up out of contact with the liquid in the container or even falling out of the container as a whole. Some existing lids include valves through which the tube must be inserted. However, such valves are only suitable for a particular size of tubing and therefore different lids are required to suit the tubes needed for different applications. Alternatively, an inventory of lids with differently sized holes must be provided.

The present invention provides a cap for use with a container holding a liquid for medical use, the cap comprising a lid portion configured to cover an opening in the container and an aperture formed in the lid portion for receiving a tube, wherein the aperture comprises a slot with a first end and a second end, defining a length between the first and second ends, and a width which is smaller than the length, wherein the slot is tapered and the width decreases from the first end to the second end.

Thus, a tube may be inserted through the aperture and moved along it until it engages with the tapered edge and becomes wedged in position. A single cap can accommodate different diameters of tube.

Preferably the cap further comprises at least one gripping feature projecting into the aperture configured to engage the surface of a tube passing therethrough. This serves to enhance the grip on the tube for more secure retention.

The gripping feature may comprise a protruding rim around the perimeter of the aperture. Preferably, the gripping feature is wedge-shaped in cross section and tapers as it projects into the aperture.

Preferably, the perimeter of the aperture is arcuate at the first and second ends and extends straight between the ends.

The lid portion may further comprises a wall extending downwardly from the perimeter of the aperture to define a guide channel. In this case, the cap may further comprise at least one gripping feature projecting into the guide channel.

Conveniently, the cap further comprises an annular collar depending from the lid portion configured for releasable attachment to a container.

The present invention also provides a cap as described above, in combination with a container holding a liquid for medical use.

### BRIEF DESCRIPTION OF THE SEVERAL VIEWS OF THE DRAWING(S)

The invention will now be described in detail, by way of example only, with reference to the accompanying drawings in which:

FIG. 1 is a plan view of a cap in accordance with the present invention;

FIG. 2 is a perspective view from above of the cap shown in FIG. 1;

FIG. 3 is a perspective view from below of the cap shown in FIG. 1;

FIG. 4 is a cross section along the line AA in FIG. 2;

FIGS. 5a, b and c show alternative cross sectional profiles of a gripping feature;

FIG. 6 is a perspective view of a larger diameter tube being inserted into the cap;

FIG. 7 is a perspective view of the larger diameter tube being gripped by the cap;

FIG. 8 is a perspective view of a smaller diameter tube being inserted into the cap; and

FIG. 9 is a perspective view of the smaller diameter tube being gripped by the cap.

### DETAILED DESCRIPTION OF THE INVENTION

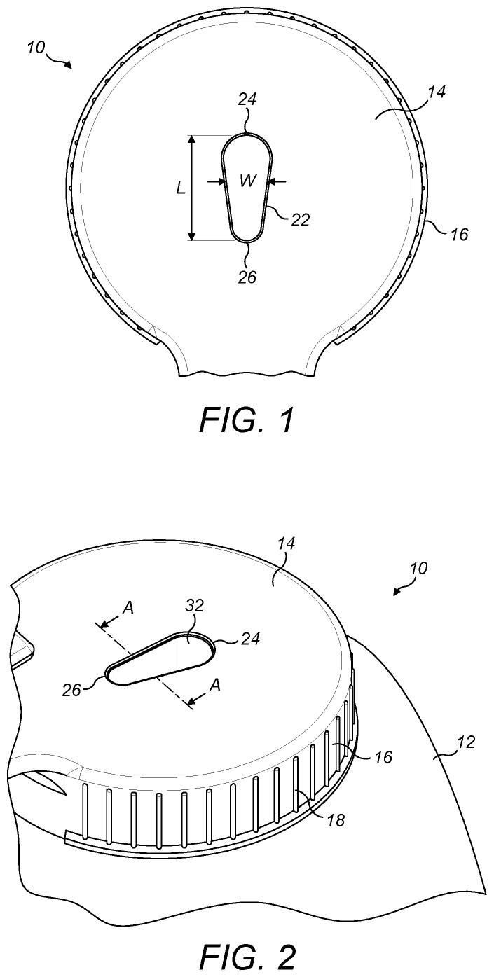

As best seen in FIGS. 1-3, one embodiment of the present invention comprises a cap 10 for a container 12 (only part of which is shown schematically in FIG. 2). The cap 10 comprises a lid portion 14 configured to cover an opening in the container. Typically, the lid portion 14 is flat and circular and connected to an annular collar 16. The collar has an exterior surface provided with ridges 18 and an internal surface provided with engagement features 20 such as a thread for releasable connection to the container 12.

An aperture 22 is defined in the lid portion 14. This may be centrally located as illustrated, or towards one side of the cap 10. The aperture 22 is in the form of a slot with first and second ends 24, 26. The aperture 22 has a length L between the ends 24, 26 and a width W. The length L is greater than the width W. Furthermore, slot tapers so that the width W decreases from the first end 24 to the second end 26. Preferably, the perimeter of the aperture 22 at the first and second ends 24, 26 is arcuate, in particular semi-circular, and extends straight between the first and second ends 24, 26.

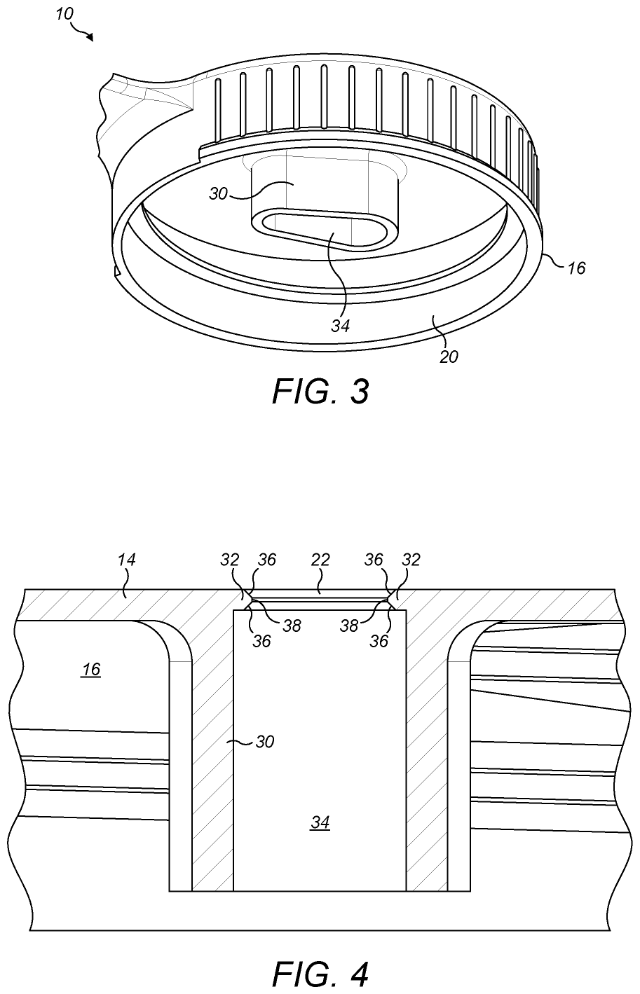

As best seen in FIG. 3, on the underside of the lid portion 14 a wall 30 extends downwardly from the perimeter of the aperture 22. The wall 30 strengthens the lid portion 14 and provides a guide channel 34 through the cap 10 for receiving a tube.

Preferably, a gripping feature 32 is also provided, which protrudes into the aperture 22 to enhance the grip on a tube passing therethrough. In this example, the gripping feature 32 comprises a rim around the perimeter of the aperture 22 which projects into the aperture 22 to define a minimum width region. As seen in FIG. 4, in cross section, the rim is wedge-shaped with sloping surfaces 36 which taper to an apex 38. Below the gripping feature 32, the wall 30 preferably extends straight down and is wider than the minimum width portion between opposing apices 38 of the gripping feature 32.

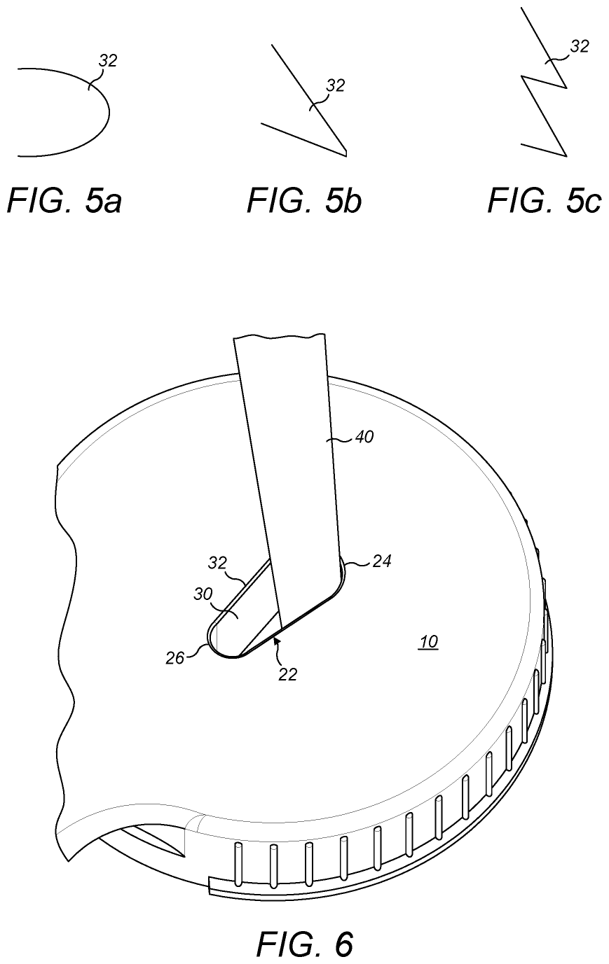

The gripping feature 32 may be configured in a different way if desired. For example, the rim around the aperture 22 may consist of a series of individual projections rather than a continuous rim. The cross sectional profile of the gripping feature 32 may also be varied. For example, as shown in FIGS. 5a, b and c, the profile may be more rounded, or a downward pointing wedge, or a saw tooth profile. The gripping feature 32 may be provided lower down on the inner surface of the wall 30 instead of or as well as round the upper perimeter of the aperture 22.

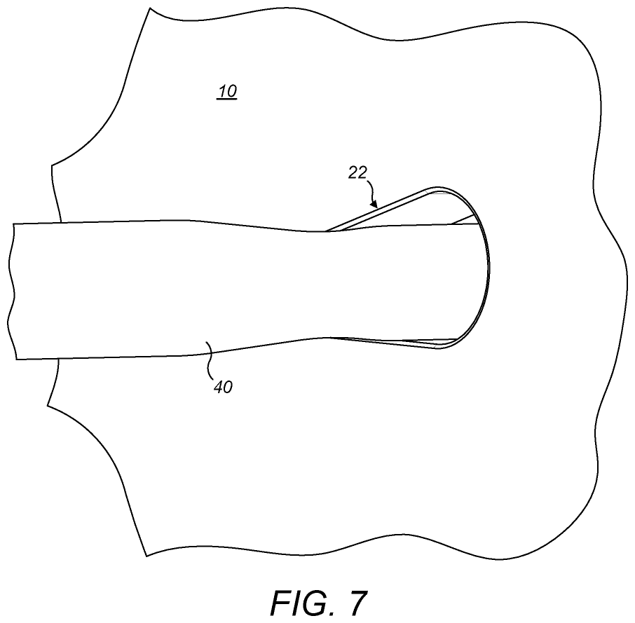

In use, the cap 10 is fitted to a container 12 and a tube 40 is passed into the container 12 through the aperture 22 and guide channel 34. When a larger diameter tube is used it is fed into the widest part of the aperture 22 at or close to the first end 24 as seen in FIG. 6. The tube 40 may then be moved sideways towards the second, narrower end 26 of the aperture 22 until it becomes wedged between the tapering edges of the aperture 22. This alone will hold the tube 40 in position, but the effect is enhanced by the gripping feature 32 engaging with the outer surface of the tube 40. The tube 40 is normally formed of a flexible material and therefore the gripping feature 32 slightly pinches into the exterior surface of the tube 40 to retain it even more firmly in place, as seen in FIG. 7. As the tube diameter is relatively large, it is gripped while only part way along the aperture 22.

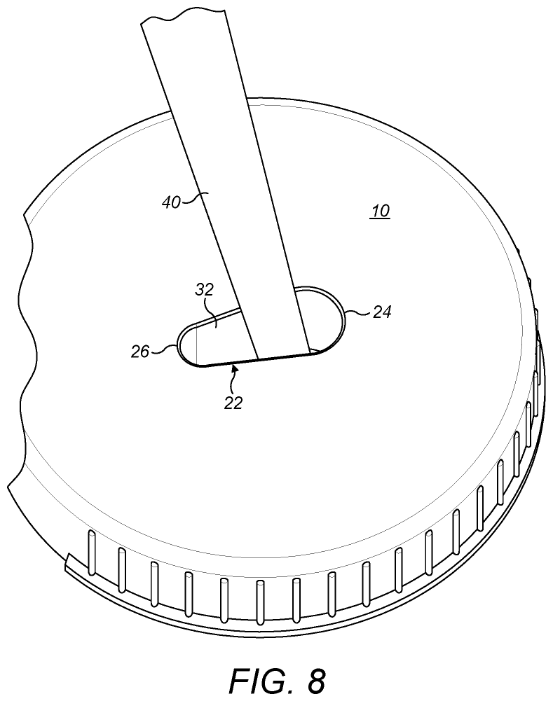

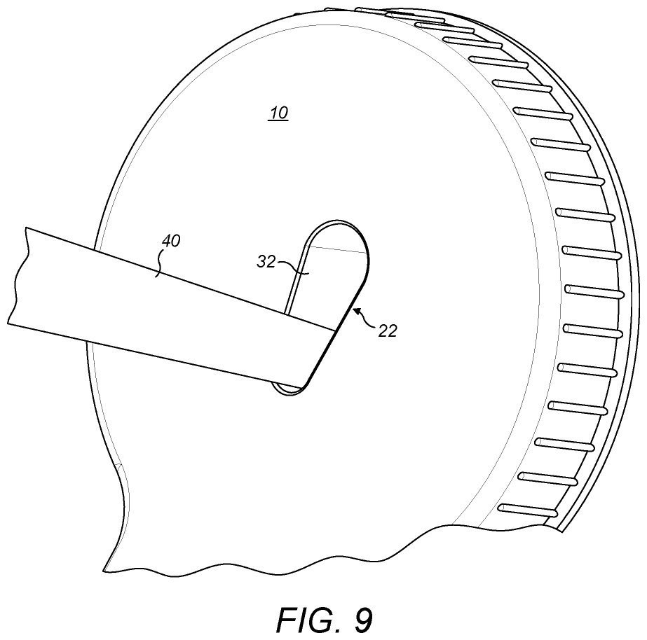

When a smaller diameter tube is used, this can be inserted in a central region of the aperture 22 and will be gripped when much closer to the narrow second end 26, as seen in FIGS. 8 and 9.

To remove the tube 40, it can be pushed back towards the wider first end 24 of the aperture 22 to release it from the gripping feature 32 and tapering edges of the aperture 22. The tube 40 can then be withdrawn up out of the container 12.

When the tube 40 is positioned in the aperture 22 in this way, the weight of the tube 40 itself, plus vibrations transmitted to the tube 40 by a pump to which it is attached, tend to cause the tube 40 to move further towards the narrower second end 26 of the aperture 22, thus increasing the grip on the tube 40. Therefore, the tube 40 will be securely retained in the cap 10 and container 12 during use.

Furthermore, a single cap 10 can be used with a variety of tubes of different diameters. Wider tubes will engage with the tapered edges and gripping feature 32 closer to the wider first end 24 of the aperture 22, whilst narrower tubes can be pushed further towards the second narrower end 26 of the aperture 22. It is therefore unnecessary for a hospital or medical facility to provide multiple caps with different sized holes.

The cap 10 is preferably made from a reusable and recyclable material, such a recyclable plastic, which can be cleaned and reused to avoid waste.

1. A cap for use with a container holding a liquid for medical use, the cap comprising a lid portion configured to cover an opening in the container and an aperture formed in the lid portion for receiving a tube, wherein the aperture comprises a slot with a first end and a second end, defining a length between the first and second ends, and a width which is smaller than the length, wherein the slot is tapered and the width decreases from the first end to the second end.

2. A cap as claimed in claim 1, further comprising at least one gripping feature projecting into the aperture configured to engage the surface of a tube passing therethrough.

3. A cap as claimed in claim 2, wherein the gripping feature comprises a protruding rim around the perimeter of the aperture.

4. A cap as claimed in claim 2, wherein the gripping feature is wedge-shaped in cross section and tapers as it projects into the aperture.

5. A cap as claimed in claim 3, wherein the gripping feature is wedge-shaped in cross section and tapers as it projects into the aperture.

6. A cap as claimed in claim 1, wherein the perimeter of the aperture is arcuate at the first and second ends and extends straight between the ends.

7. A cap as claimed in claim 1, wherein the lid portion further comprises a wall extending downwardly from the perimeter of the aperture to define a guide channel.

8. A cap as claimed in claim 2, wherein the lid portion further comprises a wall extending downwardly from the perimeter of the aperture to define a guide channel.

9. A cap as claimed in claim 7, further comprising at least one gripping feature projecting into the guide channel.

10. A cap as claimed in claim 8, further comprising at least one gripping feature projecting into the guide channel.

11. A cap as claimed in claim 1, further comprising an annular collar depending from the lid portion configured for releasable attachment to a container.

12. A cap as claimed in claim 1, in combination with a container holding a liquid for medical use.
